(19) United States
(12) Patent Application Publication
(10) Pub. No.: US 2023/0168219 A1
(43) Pub. Date: Jun. 1, 2023

(54) ELECTROCHEMICAL-SENSING APPARATUS AND METHOD THEREFOR

(71) Applicant: CARDIAI TECHNOLOGIES LTD., Calgary (CA)

(72) Inventors: Nikhil Suresh VASTAREY, Calgary (CA); Kartikeya MURARI, Calgary (CA); Raman KOUL, Calgary (CA); Anmol Singh KAPOOR, Calgary (CA); Savitri BUTTERWORTH, Calgary (CA); Jonas TEIXEIRA, Calgary (CA)

(21) Appl. No.: 17/920,359

(22) PCT Filed: Apr. 21, 2021
(86) PCT No.: PCT/CA2021/050549

§ 371 Date: Oct. 20, 2022

Related U.S. Application Data
(60) Provisional application No. 63/013,426, filed on Apr. 21, 2020.

(51) Int. Cl. G01N 27/327; G01N 27/416; G01N 33/543

(52) U.S. Cl. CPC G01N 27/3273; G01N 27/3274; G01N 27/4163; G01N 33/5438

(57) ABSTRACT

An electrochemical-sensing apparatus for analyzing a sample of a user. The apparatus has a housing with a port for receiving an electrochemical-sensor structure having a counter electrode (CE), a reference electrode (RE), and at least one working electrode (WE) for contacting the sample, and an analysis circuitry for coupling to the electrodes for analyzing biomarkers in the sample, and an output for outputting an analytical result. The analysis circuitry has a circuit for generating an excitation signal and applying it to CE and RE, at least one frequency analyzer for receiving a return signal from the at least one WE for analyzing the sample, and a set of switches for short-circuiting CE and RE and for engaging at least one calibration resistor to CE/RE and the at least one frequency analyzer for directing a calibration signal to the at least one frequency analyzer component for calibration.

### CROSS-REFERENCE TO RELATED APPLICATIONS

This application claims the benefit of U.S. Provisional Pat. Application Serial No. 63/013,426, filed Apr. 21, 2020, the content of which is incorporated herein by reference in its entirety.

### FIELD OF THE DISCLOSURE

The present disclosure relates generally to a portable electrochemical-sensing system and method for analyzing and monitoring a user’s health conditions, and in particular, to a portable electrochemical-sensing system having a point-of-care (PoC) device and disposable electrochemical-sensor structures for analyzing and monitoring a user’s health conditions by detecting one or more biomarkers and/or or disease-analytes in the patient’s biological samples received onto the electrochemical-sensor structure.

### BACKGROUND

The focus of diagnostic medicine has shifted from hospital-based testing to simple home-based testing and has resulted in patient’s increased awareness of their lifestyle. For example, portable health-monitoring devices such as blood-pressure monitors, blood-glucose meters, smartwatches with heart-rate monitors, and the like, have been widely used by patients to monitor their health conditions without going to clinics, medical labs, and/or hospitals for testing and diagnosis. Such portable health-monitoring devices enable home-based testing and significantly save patient’s time for visiting doctors and medical labs, thereby improving their quality of life. Such portable health-monitoring devices also significantly save the resources of clinics, medical labs, and hospitals.

Portable health-monitoring devices may be hand-held devices allowing a convenient analysis of a user’s health conditions. Examples of such portable health-monitoring devices include Ascensia™ BREEZE™ diabetes care system (Ascensia and BREEZE are trademarks of Ascensia Diabetes Care Holdings AG of Basel, Switzerland) and the GLUCOMETER ELITE® blood-glucose meter (GLUCOMETER ELITE is a trademark of Ascensia Diabetes Care Holdings AG of Basel, Switzerland).

Some types of portable health-monitoring devices such as blood-glucose meters, diagnose and monitor patients’ health conditions by detecting and measuring the quantity of a biomarker (such as glucose) or disease-analyte (such as protein, nucleic acid molecules, ionic metabolites, and the like) in samples of a patient’s bodily fluids. A biomarker is one or more specific compounds in a patient’s bodily fluids or more generally biological samples that are indicative of certain health conditions.

There exist a plurality of biomarkers in human biological samples. However, in a home-based testing environment, there usually is a limited amount of biological samples available for a portable health-monitoring device to process. Furthermore, the quantity of a particular biomarker in a fluid sample may be very low. Therefore, it is always a challenge for a portable health-monitoring device and sampling structure to collect, detect, and measure biomarkers found in biological samples with sufficient accuracy for determining the patient’s health condition.

Moreover, while existing portable health-monitoring devices such as a glucose-monitoring device can only detect a single biomarker from a bodily fluid sample, there exists a need for a portable health-monitoring device capable of detecting more than one biomarker for patients’ convenience and for reducing the patients’ healthcare costs.

There is also a need for diagnostic bio-sensing devices (also denoted as point-of-care (PoC) devices hereinafter) to reduce the burden on the existing healthcare system and to improve patient access to healthcare. Moreover, there is a demand for the development of PoC devices used by untrained consumers for home-based testing of physiological fluids for effectively diagnosing or predicting disease and for enhancing disease management. More particularly, there is a high demand for on-demand, portable, reliable, intuitive, and low-cost PoC devices for home-based testing for disease diagnosis and prognoses.

For example, the standard of care for heart failure in the art is retroactive rather than proactive in delivering healthcare services. After being diagnosed with heart failure, patients usually have to routinely visit their healthcare provide (HCP) for lab testing which is a time consuming burden on the patients and ineffective as the patients may be at risk between visits.

While sorely needed, a portable, at-home testing is only part of a complete solution. To have any meaningful impact on a patient’s health, especially in times of emergency, the patient needs to have an option to access emergency health care services as, for example, emergent issues such as heart failure may lead to progressively debilitating conditions and sudden life-threatening events.

Infectious diseases originating from causative agents (also called “infectious agents” or “infectious vectors”) such as bacteria and/or viruses can lead to acute and chronic infections in animals and humans thereby often posing a huge risk to overall human and animal health. Such infectious diseases include but not limited to diseases related to respiratory systems, digestive systems, circulatory systems, and nervous systems originating from infectious agent/vector. An example of such infectious diseases is flu (also commonly referred to as influenza) caused by one of several related “RNA viruses” (i.e., viruses whose genetic material is RNA) of the Orthomyxoviridae family, and characterized by fever, headache, fatigue, and other symptoms.

Other examples include sever acute respiratory syndrome (SARS), Middle East respiratory syndrome (MERS-CoV), novel coronavirus (2019-nCoV or SARS-CoV-2) which caused the COVID-19 pandemic; hereinafter, the terms “coronavirus”, “SARS-CoV 2” and “COVID-19” may be used interchangeably), hepatitis, plague, and the like. Some of the infectious diseases are highly contagious and may lead to an epidemic which may spread quickly across continents and then to a pandemic over the entire world. Such pandemic events have huge impacts on societies globally.

A historical example of a pandemic is the “bubonic plague” which killed millions of peoples across the world. In 2003, the SARS outbreak killed at least 1,000 people, infected about 8,000 people across the Asia-Pacific region, and caused disruption in travel and closure of workplaces with an overall economic loss of about $40 billion. While some vaccines have become available for limiting the spread of specific infectious diseases, rapid detection and diagnosis are generally the keys to stop the spread of an infectious disease.

For example, the highly contagious COVID-19 outbreak has caused a huge public health issue over the world. However, its clinical characteristics and epidemiology currently remain largely unclear thereby limiting the ability to fully characterize the disease spectrum. Moreover, unlike other coronavirus infections, the incubation period of COVID-19 varies greatly (usually less than 24 days).

Hitherto while efforts to contain SARS-CoV-2 are ongoing, given the many uncertainties regarding pathogen transmissibility and virulence, the effectiveness of these efforts is unclear. The fraction of undocumented but infectious cases is a critical epidemiological characteristic to be determined. These undocumented infections often experience mild, limited, or no symptoms and hence go unrecognized, which, depending on their contagiousness and numbers, can expose a far greater portion of the population to SARS-CoV-2 than would otherwise occur. The mathematical models that simulates the spatiotemporal dynamics of infections among different populations have not shown a clear vision to what extend the disease will spread and when business activities and travel can get safely return to normal.

Thus, there is a pressing need in public-health area of new technologies for rapid detection, diagnosis, and monitoring of infections such as the SARS-CoV-2 infection, to enable outbreak management which may monitor each individual from the initiation of the infection to the stage of full recovery in order to develop effective governmental strategies for screening the level of outbreak, entire public health screening, identify the sources of transmission, and safely resuming business activities.

Therefore, a solution is needed for improving emergency access involving patient’s location, historical patient data, and communication when a patient is unresponsive. However, current diagnostic devices are limited in communicating with emergency services even in a situation that requires immediate medical attention, which may put patients in life-threatening risks in emergent situations.

Moreover, it is highly desirable for improved, efficient, and rapid methods for the detecting and identifying infectious agents that cause diseases such as influenza, respiratory diseases, SARS-Cov-2, sexually transmitted diseases, blood diseases, viral diseases, bacterial diseases, and/or the like. Moreover, there exists a need of rapid and quantitative serological assays with portability, ultra-sensitivity, and reasonable throughput for detection of COVID-19 in order to timely diagnose the disease.

U.S. Pat. Application Publication No. 2011/0089957 A1 to Sheppard Jr. teaches arrays of biosensors along with methods for operating the arrays of biosensors. The array of biosensors may include a first reference electrode that is connected to an input of a first control amplifier; a first working electrode and a second working electrode in proximity with the first reference electrode; and a counter electrode that is connected to at least an output of the first control amplifier, where the first control amplifier is operative with the counter electrode to maintain a first specified voltage between the first working electrode and the first reference electrode, and between the second working electrode and the first reference electrode. The array of biosensors optionally may further include a second reference electrode that is connected to an input of a second control amplifier, where the second control amplifier is operative with the counter electrode to maintain a second specified voltage between the first working electrode and the second reference electrode, and between the second working electrode and the second reference electrode.

Canadian Patent Application Ser. No. 2,940,150 teaches methods for detecting a hydrogen leak and quantifying a rate of the same in a polymer electrolyte membrane fuel cell stack, as well as a fuel cell diagnostic apparatus that diagnoses a hydrogen leak in a fuel cell stack.

### SUMMARY

Embodiments disclosed herein relate to a portable electrochemical-sensing system and method for detection of infectious disease agents and/or for analyzing and monitoring a user’s health conditions. In some embodiments, the portable electrochemical-sensing system uses biosensors for detecting the presence of one or more analytes or biomarkers from body fluids. The system disclosed herein allows a rapid detection of infection/infectious disease via one or more detection routes, simultaneously from varied biological samples such as cells, tissues, bodily fluids, and/or the like.

As those skilled in the art will understand, an analyte is a chemical component, constituent, or species that is of interest in an analytical procedure being conducted on the biological samples. The term “analyte” often refers to relatively simple elements or molecules such as serum chloride or liver enzymes that are detectable in an analytic process.

Those skilled in the art will also understand that a biomarker is biological molecule typically found in blood, other body fluids, or tissues that may be used as a sign of a normal or abnormal process, or of a condition or disease. A biomarker has a detectable characteristic that may be objectively measured and evaluated as an indicator of normal biologic processes, pathogenic processes, or pharmacologic response to a therapeutic intervention. The term “biomarker” often refers to markers for detecting or diagnosing specific diseases or groups of diseases which may be malignant lesions or non-malignant diseases such as cardiovascular disease.

Notwithstanding the above differences, those skilled in the art will appreciate that the electrochemical-sensing system and method described herein may be adapted to detect suitable analytes and/or biomarkers in various embodiments. Therefore, in the description hereinafter, the terms “analyte” and “biomarker” may be used interchangeably.

According to one aspect of this disclosure, a portable electrochemical-sensing system comprises a point-of-care (PoC) device and disposable electrochemical-sensor structures (such as disposable sensing strips) for analyzing and monitoring the health conditions of a user or patient.

In some embodiments, the PoC device collaborates with the disposable electrochemical-sensor structure for detecting and collecting information from biomarkers found in mammalian biological samples.

In some embodiments, the electrochemical-sensor structure comprises a sample-receiving region for receiving a patient’s biological samples. The electrochemical-sensor structure may be inserted into otherwise coupled to the PoC device. The PoC device then detects and measure the quantity of one or more biomarkers and/or or disease-analytes indicative of health conditions in the received biological samples by measuring the electrochemical properties thereof. In some embodiments, analyte concentrations are quantified electrochemically and noise from undesirable proteins is reduced by the introduction of a filtration unit.

With the portable electrochemical-sensing system disclosed herein, it may be possible to diagnose certain illnesses without an in-person meeting with a physician, and the user may avoid making a visit to the clinic or hospital for simple diagnostic tests such as finger-prick blood tests, thereby reducing the user’s wait time at clinics and the time spent by healthcare professionals for performing such simple diagnostic tests.

The portable electrochemical-sensing system disclosed herein is suitable for use by untrained users for home-based testing of physiological fluids for effectively diagnosing or predicting diseases and for enhancing disease management. The portable electrochemical-sensing system disclosed herein is suitable for use by health workers and professionals.

The portable electrochemical-sensing system disclosed herein is efficient in monitoring patient’s health conditions by detecting one or more analytes in the biological samples received onto the electrochemical-sensor structure. Related methods and components of the portable electrochemical-sensing system for precisely detecting the analytes are also disclosed.

According to one aspect of this disclosure, the portable electrochemical-sensing system comprises a PoC device acting as a reader and a sensor strip.

In various embodiments, the electrochemical-sensor structure may comprise single or multiple working electrodes (WE) along with corresponding counter and reference electrodes (CE and RE respectively). The electrochemical-sensor structure is connected to a portable PoC device. The PoC device may detect the electrochemical properties of the biological samples from the sample-receiving region of the electrochemical-sensor structure, to produce a signal comprising a fluid reading wherein the fluid reading is related to the electrochemical properties of an analyte in the biological samples thereby indicating the presence, absence, or the quantity of analyte in the biological samples.

The PoC devices disclosed herein measure the quantity of specific biomarkers in biological samples that are indicative of health conditions. By using the PoC device, a user may diagnose certain illnesses without an in-person meeting with a physician.

According to one aspect of this disclosure, there is provided an electrochemical-sensing apparatus for analyzing a sample of a user. The apparatus comprises: a housing comprising at least one first port for receiving an electrochemical-sensor structure, the electrochemical-sensor structure comprising a first set of electrodes for contacting the sample, the first set of electrodes comprising a counter electrode (CE), a reference electrode (RE), and at least one working electrode (WE); an analysis circuitry for electrically coupling to the first set of electrodes of the electrochemical-sensor structure for analyzing one or more biomarkers in the sample; and an output for outputting an analytical result of said analysis of the one or more biomarkers in the sample. The analysis circuitry comprises: an excitation-signal circuit for generating an excitation signal and applying the excitation signal to the CE and RE, at least one frequency analyzer component for receiving a return signal from the at least one WE in response to the excitation signal for analyzing the sample, and a set of switches for short-circuiting the CE and RE and for engaging at least one calibration resistor to the short-circuited CE and RE and the at least one frequency analyzer component for directing a calibration signal to the at least one frequency analyzer component for calibration.

According to one aspect of this disclosure, there is provided an electrochemical-sensing system for analyzing a sample of a user. The system comprises: an electrochemical-sensor structure comprising a first set of electrodes for contacting the sample, the first set of electrodes comprising a CE, a RE, and at least one WE; an electrochemical-sensing apparatus comprising: a housing comprising at least one first port for receiving the electrochemical-sensor structure, and an analysis circuitry for electrically coupling to the first set of electrodes of the electrochemical-sensor structure for applying an excitation signal sweeping a predefined first frequency range to the sample and receiving a response signal for analyzing one or more biomarkers in the sample, and an output for outputting an analytical result of said analysis of the one or more biomarkers in the sample; and a prediction module for using an artificial-intelligence (AI) method for predicting a response signal in response of the excitation signal sweeping a predefined second frequency range. The second frequency range is lower than the first frequency range.

According to one aspect of this disclosure, there is provided a circuitry for analyzing one or more biomarkers in a sample of a user; the circuitry comprises: a coupling CE, a coupling RE, and one or more coupling WEs; an excitation-signal circuit for generating an excitation signal and applying the excitation signal to the coupling CE and the coupling RE; one or more signal analyzers each electrically connected to a respective one of the one or more coupling WEs for receiving a return signal from the respective coupling WE in response to the excitation signal for analyzing the one or more biomarkers; at least one calibration resistor; and a set of switches each switchable between an OPEN state and a CLOSED state; the set of switches are configured for, when in the CLOSED states, electrically connecting the coupling CE and the coupling RE and electrically connecting the one or more signal analyzers to the connected coupling CE and coupling RE via the at least one calibration resistor for directing a calibration signal to the at least one frequency analyzer component for calibration; and the set of switches are configured for, when in the OPEN states, electrically disconnecting the coupling CE from the coupling RE and electrically disconnecting the one or more signal analyzers from the coupling CE and the coupling RE for analyzing the one or more biomarkers.

In some embodiments, the set of switches are synchronously switchable between the OPEN state and the CLOSED state.

In some embodiments, the at least one calibration resistor is a single calibration resistor.

In some embodiments, the at least one calibration resistor comprise a plurality of calibration resistors.

In some embodiments, the plurality of calibration resistors have a same resistance.

In some embodiments, at least a first subset and a second subset of the plurality of calibration resistors have different resistances.

In some embodiments, the one or more signal analyzers are electrically connected to the one or more coupling WEs via one or more first amplifiers with each signal analyzer electrically connected to the respective WE via a respective one of the one or more first amplifiers.

In some embodiments, the set of switches are configured for, when in the CLOSED state, electrically connecting the coupling CE and the coupling RE and electrically connecting the one or more first amplifiers to the connected coupling CE and coupling RE via the at least one calibration resistor.

In some embodiments, the circuitry further comprises at least one first frequency generator for providing one or more control signals to the one or more signal analyzers.

In some embodiments, the at least one first frequency generator is configured for generating the one or more control signals of various frequencies within a predefined sweeping frequency-band.

In some embodiments, the excitation-signal circuit comprises a second frequency generator for generating the excitation signal.

In some embodiments, the second frequency generator comprises a first one of the one or more frequency-response analyzers.

In some embodiments, the excitation-signal circuit further comprises a frequency filter for filtering an output of the second frequency generator for generating the excitation signal.

In some embodiments, the excitation-signal circuit further comprises a microcontroller for controlling the filter and the second frequency generator.

In some embodiments, the excitation-signal circuit further comprises a second amplifier for amplifying an output of the frequency filter for generating the excitation signal.

According to one aspect of this disclosure, there is provided an electrochemical-sensing apparatus for analyzing a sample of a user; the apparatus comprises: an analysis circuitry as described above; and an output for outputting an analytical result of said analysis of the one or more biomarkers in the sample.

In some embodiments, the electrochemical-sensing apparatus further comprises a housing comprising at least one first port for receiving an electrochemical-sensor structure, the electrochemical-sensor structure comprising a first set of electrodes for contacting the sample, the first set of electrodes comprising a CE for coupling with the coupling CE, a RE for coupling with the coupling RE, and one or more WEs for coupling with the one or more coupling WEs.

According to one aspect of this disclosure, there is provided an electrochemical-sensing system for analyzing a sample of a user; the system comprises: an analysis circuitry for applying to the sample an excitation signal sweeping a predefined first frequency range and receiving a response signal for analyzing one or more biomarkers in the sample; an output for outputting an analytical result of said analysis of the one or more biomarkers in the sample; and a prediction module for using an AI method for predicting a response signal in response of the excitation signal sweeping a predefined second frequency range.

In some embodiments, the AI method comprises a deep neural network (DNN).

In some embodiments, the second frequency range is lower than the first frequency range.

In some embodiments, the electrochemical-sensing system further comprises: an electrochemical-sensing apparatus comprising a housing receiving therein the analysis circuitry, the housing comprising a display for displaying the output and at least one first port for receiving an electrochemical-sensor structure, the electrochemical-sensor structure comprising a first set of electrodes for contacting the sample, the first set of electrodes comprising a CE for coupling with the coupling CE, a RE for coupling with the coupling RE, and one or more WEs for coupling with the one or more coupling WEs.

In some embodiments, the electrochemical-sensing system further comprises: a computing device for communicating with the electrochemical-sensing apparatus, the computing device comprising the prediction module.

In some embodiments, the electrochemical-sensing apparatus further comprises the prediction module received in the housing.

According to one aspect of this disclosure, there is provided an electrochemical-sensing system for analyzing a sample of a user; the system comprises: an analysis circuitry for applying to the sample an excitation signal sweeping a predefined first frequency range and receiving a response signal for analyzing one or more biomarkers in the sample; an output for outputting an analytical result of said analysis of the one or more biomarkers in the sample; and a prediction module for using an artificial-intelligence (AI) method for predicting steady-stage measurement data based on a portion of the response signal.

In some embodiments, the portion of the response signal is a beginning portion of the response signal before the response signal reaches a steady stage.

In some embodiments, the AI method comprises a deep neural network (DNN).

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced designate corresponding parts throughout the different views.

FIGS. 1A and 1B are schematic perspective and plan views, respectively, of a portable electrochemical-sensing system according to some embodiments of this disclosure, the portable electrochemical-sensing system comprising a portable diagnostic electrochemical-sensing apparatus and a disposable electrochemical-sensor structure;

FIG. 2A is a perspective view of the electrochemical-sensor structure of the portable electrochemical-sensing system shown in FIG. 1A;

FIG. 2B is a cross-sectional view of the electrochemical-sensor structure shown in FIG. 2A along the cross-sectional line A-A;

FIG. 2C is a cross-sectional view of the electrochemical-sensor structure shown in FIG. 2A along the cross-sectional line B-B;

FIG. 2D is a cross-sectional view of the electrochemical-sensor structure shown in FIG. 2A along the cross-sectional line C-C;

FIG. 2E is a schematic plan view of the electronic structure of the electrochemical-sensor structure shown in FIG. 2A, the electrochemical-sensor structure comprising a plurality of electrodes;

FIG. 3A is a schematic view of the electrochemical-sensor structure shown in FIG. 2A having a substrate and a plurality of electrodes including a reference electrode (RE), a control electrode (CE), and a working electrode (WE);

FIG. 3B is a schematic view of the electrochemical-sensor structure shown in FIG. 2A, illustrating the substrate and the WE, wherein the WE comprises a nanostructured-sensing surface having zinc oxide (ZnO) nano-rods;

FIG. 3C is a schematic view of the electrochemical-sensor structure shown in FIG. 2A, illustrating the substrate and the WE, wherein the WE comprises a nanostructured-sensing surface embedded with ZnO;

FIG. 4A is a schematic diagram of an analysis circuitry of the portable diagnostic electrochemical-sensing apparatus of the portable electrochemical-sensing system shown in FIG. 1A;

FIG. 4B is a circuit diagram of an example of the analysis circuitry shown in FIG. 4A;

FIG. 4C is a circuit diagram of an example of a quick-charging circuit connecting a microcontroller and a frequency filter of the analysis circuitry shown in FIG. 4A;

FIG. 5A is a schematic diagram of the analysis circuitry shown in FIG. 4A configured in a first, calibration phase;

FIG. 5B is a schematic diagram of the analysis circuitry shown in FIG. 4A configured in a second, measurement phase;

FIG. 6 is a schematic diagram of an analysis circuitry of the portable diagnostic electrochemical-sensing apparatus of the portable electrochemical-sensing system shown in FIG. 1A, according to some alternative embodiments;

FIG. 7 is a schematic diagram of an analysis circuitry of the portable diagnostic electrochemical-sensing apparatus of the portable electrochemical-sensing system shown in FIG. 1A, according to some alternative embodiments;

FIG. 8A is a time-domain signal diagram of an example of an excitation signal generated by the analysis circuitry shown in FIG. 4A;

FIG. 8B is time-domain signal diagram of a response signal received by the analysis circuitry shown in FIG. 4A in response to the excitation signal shown in FIG. 8A;

FIG. 9 is a schematic diagram of a process for predicting steady-stage measurement data using a machine learning method;

FIG. 10 is a schematic diagram of a deep neural network (DNN) based AI prediction engine used by the process shown in FIG. 9 for deep learning and for predicting the steady-stage measurement data;

FIG. 11 is a schematic diagram of a portable electrochemical-sensing system, according to some embodiments of the present disclosure;

FIG. 12 is a schematic diagram showing a simplified hardware structure of a computing device of the portable electrochemical-sensing system shown in FIG. 11; and

FIG. 13 a schematic diagram showing a simplified software architecture of a computing device of the portable electrochemical-sensing system shown in FIG. 11.

### DETAILED DESCRIPTION

### Overview

Embodiments disclosed herein generally relate to a portable electrochemical-sensing system for monitoring a user’s health conditions. In some embodiments, the portable electrochemical-sensing system comprises diagnostic bio-sensing device and a sampling structure such as a disposable electrochemical-sensor structure in the form of a sensing strip, for monitoring a patient’s health conditions by detecting various analyte such as proteins and other molecules in the patient’s biological samples (or simply denoted “samples”) received onto the electrochemical-sensor structure. The presence, absence, or variation in the quantities of certain analyte in biological samples may be used as an indicator or predictor of disease.

In some embodiments, the electrochemical-sensor structure comprises a sample-receiving region for receiving the biological samples. The sample-receiving region of the electrochemical-sensor structure may comprise a substrate with a plurality of electrodes and having one or more detection elements thereon suitable for detecting one or more analyte.

In some embodiments, the substrate may be made of a flexible polymeric material such as a flexible modified/unmodified (treated or untreated) acrylic or polymer membrane strip with one or more detection elements thereon for detecting one or more analyte.

In some embodiments, the PoC device may comprise one or more potentiostat circuitries for monitoring the electrochemical reaction between the analyte in the biological samples and the detection elements.

The potentiostat circuitries may comprise a DC potentiostat circuitry whose application can be confined to chronoamperometry and voltammetry, when used in combination with a frequency response analyzer, may be used as an impedance-analysis system.

In particular, the components of the system disclosed herein may be used to stimulate the biological samples with an AC, DC, or a combination of thereof. In some embodiments, the signal may constitute an AC amplitude with a specific frequency offset with a DC signal. The inspecting signal may also be generated in different combinations. For instance, an embodiment may simply use a DC signal for sample inspection resulting in a flow of current in either direction, thereby allowing for characterization, recognition, or analysis of the substrate. More specifically, the system uses a range of frequencies to gauge criteria related to, but not limited to, quality of substrate, conductance of the electrode, quality of the biosensor immobilized on the electrode, and binding efficiency of the analyte to the biosensor.

In some embodiments, the diagnosis of the system through electrochemical impedance spectroscopy (EIS) may be done through domain recognition aided by the resultant Nyquist-plot analysis. For instance, by relying on Nyquist-plot pre-characterization of the capture ligand on strips, newly scanned data may be used in comparison to gauge the quality of the immobilized layers after a certain duration in storage, or prior to use.

When biological samples are placed on the sample-receiving region of the electrochemical-sensor structure, electrochemical interaction between the analyte in the biological samples and the detection elements occur and cause the electrochemical properties to change. The electrochemical-sensor structure is engaged with an ex vivo PoC device which imparts energy to the biological samples and measures the electrochemical properties thereof for generating a reading indicative of the concentration of a specific compound in the biological samples. The imparted energy may be electrical energy and the measured electrochemical property may be the potential difference, current, impedance, and/or the like.

As an analyte often possesses an affinity and specificity to a particular detection element, an electrochemical-sensor structure generally needs to be specifically manufactured for detection of a particular type of analyte.

Antibodies, nucleic acid aptamers and enzymes are often used as detection elements of bio-sensing devices because of their high specificity and affinity for respective biomarkers. Given the high specificity of a detection element to a particular analyte, the sample-receiving region of the electrochemical-sensor structure 104 may only contain one type of detection element and may be used to detect a single analyte. Moreover, different analytes possess different electrochemical properties. Accordingly, a PoC device needs to be calibrated with respect to a particular analyte in order to measure the electrochemical properties thereof. Therefore, in some embodiments, the PoC device for measuring multiple analyte may comprise a calibration functionality for adjusting the settings thereof for adapting to each of the multiple analyte.

In some embodiments, one or more potentiostat circuitries may be calibrated by using diluted human plasma/serum/blood/fluid samples with known concentrations of targeted disease-analyte, obtained anonymously from suitable sources such as medical labs. The potentiostat circuitries of the PoC device may then be calibrated using samples of different analyte concentrations.

Detection of analyte binding signal can be based on electrochemical signals, optical signals (such as chemiluminescence, reflectance, and/or the like), or magnetic transduction signals. Such electrochemical detection methods rely on either voltage or current to detect analyte binding and are suitable for implementation in miniaturized electrical biosensor devices. These methods monitor the change in electrical impedance that occurs when an analyte binds to the capture ligand which is then correlated to the concentration of the target analyte.

In some embodiments, the PoC device uses an identification element on the electrochemical-sensor structure or on the carrying vial thereof for determining the biomarker to be analyzed. The identification element may include detection electrodes, radio frequency identification (RFID) tags, one-dimensional barcodes, two-dimensional barcodes such as Quick Response (QR) codes, and/or the like.

### Description of Various Embodiments

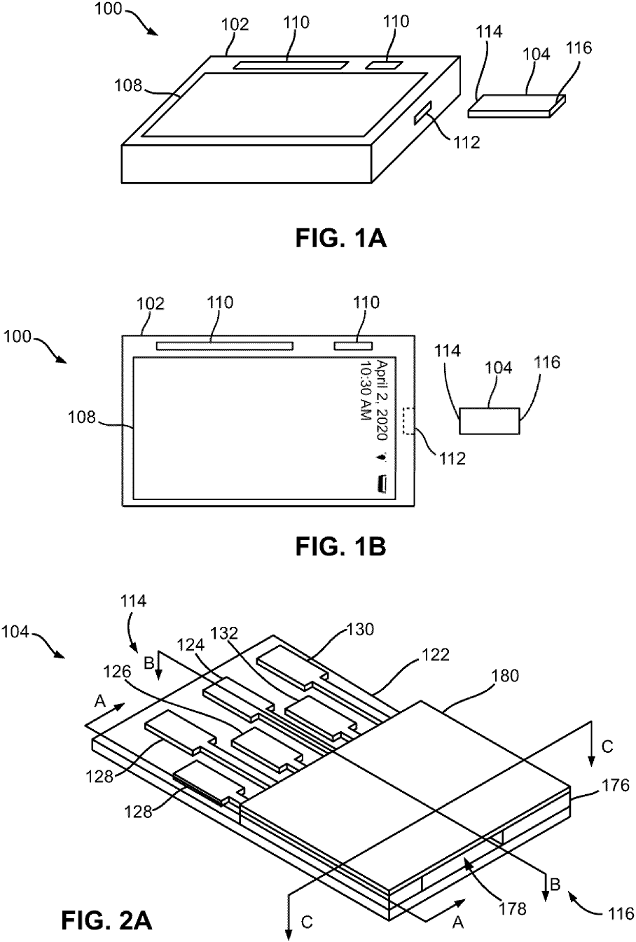

Turning now to FIGS. 1A and 1B, a portable electrochemical-sensing system is shown and is generally identified using the reference numeral 100, which may be used for analyzing, determining, and monitoring a user’s health conditions, including infection by an infectious disease such as SARS, MERS-CoV, SARS-CoV-2, and/or the like.

The portable electrochemical-sensor system 100 may be used for home-based testing for disease diagnosis and prognosis. However, those skilled in the art will appreciate that the portable electrochemical-sensing system 100 may also be used in other suitable places such as health centers, clinics, hospital, and the like.

As shown in FIGS. 1A and 1B, the portable electrochemical-sensing system 100 in these embodiments comprises a portable diagnostic electrochemical-sensing apparatus 102 in the form of a point-of-care (PoC) device with a size suitable for personal use and a disposable electrochemical-sensor structure 104, for analyzing, determining, and monitoring user’s health conditions by detecting various analyte in the patient’s biological samples such as bodily fluid samples received onto the electrochemical-sensor structure. The detection of the analyte may be used for detecting infectious agents and assessing the patient’s health conditions with respect to an infectious disease, wherein the presence, absence, or variation in the quantities of a certain analyte in biological samples may be used as an indicator or predictor of disease. Herein, infectious agents or infectious vectors may be nucleic acids, blood-bom vectors, zoonotic diseases, microbes (such as bacteria, viruses, fungi, protozoa, and/or the like), helminths, host immunoglobulins, and/or the like.

Many aspects of the portable diagnostic apparatus 102 and the electrochemical-sensor structure 104 may be similar to those disclosed in Applicant’s Canadian Patent Application Ser. No. 3,060,849, entitled “PORTABLE ELECTROCHEMICAL-SENSOR SYSTEM FOR ANALYZING USER HEALTH CONDITIONS AND METHOD THEREOF”, filed on Nov. 04, 2019, the content of which is incorporated herein by reference in its entirety.

In particular, the PoC device 102 in these embodiments comprises a screen 108, a user-input structure for receiving user inputs, a strip-receiving port 112 for receiving a proximal side 114 of the electrochemical-sensor structure 104, a control circuitry having a control structure (not shown) such as a RFduino microcontroller offered by RFduino Inc. of Hermosa Beach, CA, USA, and relevant circuits. The PoC device 102 also comprises a power source such as battery for powering various components. As will be described in more detail later, the PoC device 102 further comprises a set of coupling electrodes in the strip-receiving port 112 for electrically engaging the electrodes of the electrochemical-sensor structure 104.

The user-input structure may comprise one or more buttons 110 and/or a touch-sensitive screen (such as a touch-sensitive screen 108 in some embodiments) for receiving user inputs such as user instructions (e.g., turning the PoC device 102 on or off, starting a diagnostic process, displaying readings obtained in the diagnostic process, displaying previous diagnostic readings, and/or the like) and/or user data (e.g., the user’s age, sex, weight, height, and/or the like).

The control circuitry may include an analysis circuitry such as a potentiostat circuitry for electrochemical-sensing (described in more detail later) and a monitoring circuitry for other tasks such as performing user-instructed operations, detecting the insertion of the electrochemical-sensor structure 104, reading and displaying the measured levels of biomarkers, storing measurement data, transmitting measurement data to a remote device for trend tracking, and/or the like. In various embodiments, the analysis circuitry and monitoring circuitry may use the same microcontroller or alternatively use separate microcontrollers.

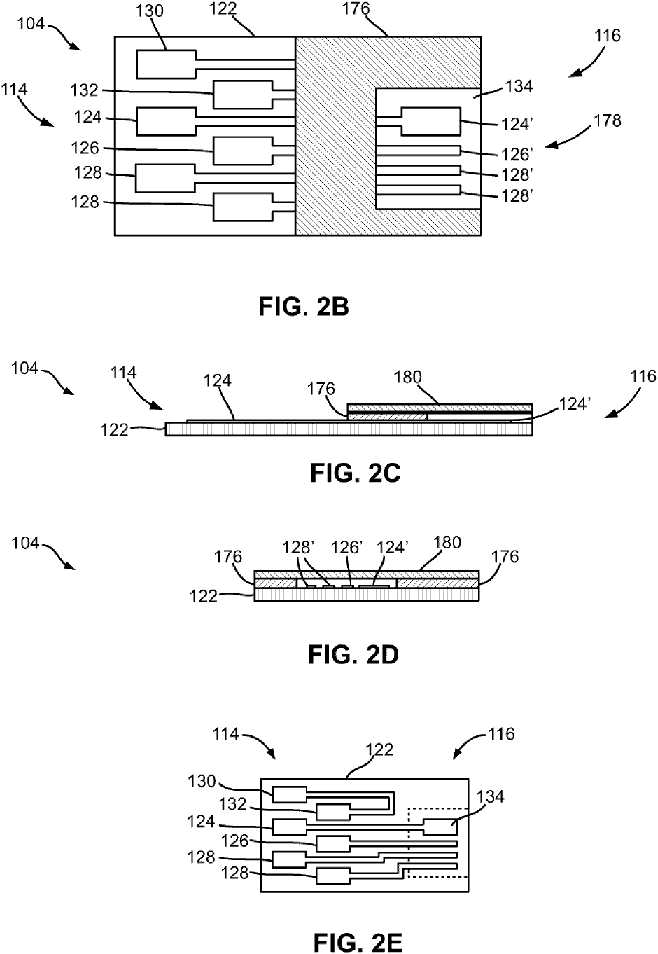

FIGS. 2A to 2E show the physical and electrochemical structures of the electrochemical-sensor structure 104 in some embodiments. As shown, the electrochemical-sensor structure 104 comprises a substrate 122, a plurality of electrodes 124 to 132 deposited, printed, or otherwise coupled to the substrate 122, and a hydrophobic middle layer 176 and a protection layer 180 about a sample-receiving region 134 (also called a sampling region) on a distal side 116 of the substrate 122.

In some embodiments, the substrate 122 may be made of a flexible material such as a flexible polyimide membrane strip. In some embodiments, the flexible substrate 122 may be made of a modified or unmodified polymeric substrate including but not limited to track-etched membranes, treated or untreated acrylic substrates, and/or the like. In some embodiments, the track-etched membrane 122 may be a porous polyimide membrane.

In some embodiments, the track-etched membrane 122 may have a porosity equal to or greater than 30%. Herein, the porosity of a material is defined as the ratio of the volume of void or empty spaces over the total volume of the material. In some embodiments, the track-etched membrane 122 may have a porosity equal to or greater than 50%.

In some embodiments, pore size, shape, and density of the track-etched membrane can be varied in a controllable manner so that a membrane with selected transport and retention characteristics can be produced. Because of the precisely determined structure of track-etched membranes, using a track-etched membrane as the substrate 122 may give rise to distinct advantages over conventional membranes. For example, in some embodiments, pore size, shape, and density of the track-etched membrane 122 may be varied in a controllable manner so that a membrane with selected transport and retention characteristics may be produced. A membrane 122 with a higher pore density allows the metal layers to be coupled thereto with coarser surfaces which in turn allows increased capacity to house a larger amount metal layers of three-dimensional (3D) nano-rods (described later) to be grown at the membrane surface. More nano-rods relate to more binding sites available for antibody molecules, which in turn increases the overall sensitivity of the electrochemical-sensor structure 104. Moreover, a membrane 122 with a higher pore density also facilitates the flow of the fluidic biological samples thereon.

In these embodiments, the electrodes 124 to 132 may be made of or comprise conductive or semi-conductive metals such as gold (Au), chromium (Cr), titanium, platinum, silver, and/or the like. The electrodes 124 to 132 are distributed on the proximal side 114 of electrochemical-sensor structure 104 and comprise a first set of electrodes including a reference electrode (RE) 124, a control electrode (CE) 126, and two working electrodes (WEs) 128 forming an analysis circuit, and a second set of electrodes including a pair of electrically connected identification-electrodes 130 and 132 forming an identification circuit. The RE 124, CE 126, and WEs 128 extend into the sample-receiving region 134 and form corresponding RE 124′, CE 126′ and WEs 128′ for measuring the electrochemical properties of biological samples (not shown) received therein. The pair of identification electrodes 130 and 132 are electrically joined by a trace with a pre-defined resistance or a pre-defined impedance, for indicating the type of biomarker or infectious agent that the electrochemical-sensor structure 104 is suitable to detect.

As shown in FIG. 2B, the distal-side electrodes RE 124′, CE 126′, and WE 128′ (corresponding to and connected to the RE 124, CE 126, and WE 128, respectively) are laterally spaced at a same distance. The electrode RE 124′ has a much larger surface than that of the electrode CE 126′ or WE 128′. For example, in some embodiments, the surface-area ratio of WE 128′, CE 126′, and RE 124′ may be about 1:1:4.

The hydrophobic middle layer 176 covers a distal portion (also identified using reference numeral 116) of the electrochemical-sensor structure 104 except the sample-receiving region 134. The hydrophobic middle layer 176 has a distal-end opening 178 forming a rear-facing sampling port for receiving biological samples into the sample-receiving region 134 and in contact with the distal-side electrodes RE 124′, CE 126′, and WE 128′. The protection layer 180 is coupled to the hydrophobic middle layer 176 and covers the distal portion 116 (including the sample-receiving region 134). In these embodiments, the protection layer 180 is made of a suitable material such as glass or plastic.

In these embodiments, the surfaces of the WEs 128′ may be modified or otherwise treated with a mediator to mediate the electron transfer from the electrodes to body fluids. Different WEs 128′ may be configured to harbor different elements for detecting one or more analyte, agents, and/or targets.

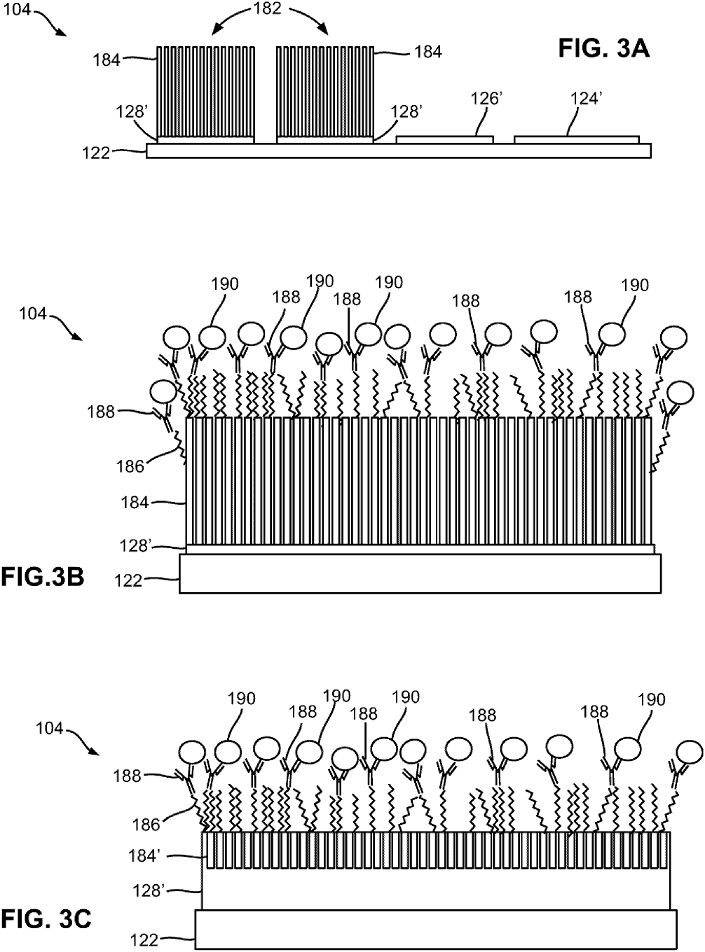

FIG. 3A is a schematic view of the electrochemical-sensor structure 104 showing the substrate 122 and the electrodes RE 124′, CE 126′, and WE 128′ in some embodiments. FIG. 3B is a schematic view of the electrochemical-sensor structure 104 showing the substrate 122 and the electrode WE 128′. As shown, the electrochemical-sensor structure 104 comprises a nanostructured-sensing surface in the sample-receiving region 134 thereof for amplifying the amount of biomarker binding to the electrochemical-sensor structure 104 in order to achieve improved sensitivity.

More specifically, the distal-side electrode WE 128′ in these embodiments comprises a nanostructured-sensing surface 182 having a plurality of nano-rods 184 such as zinc oxide (ZnO) nano-rods. In some embodiments, the ZnO nano-rods may be synthesized by depositing ZnO onto the distal-side electrode WE 128′ on the substrate (acting as seeds) and then immersing the substrate consisting the coated electrode in a chemical bath consisting of zinc nitrate hexahydrate and hexamethyline tetramine at a temperature below the boiling point of water and preferably about 80° C. for “growing” the ZnO nano-rods.

The nano-rods 184 are coated with a specific type of detection element 188 such as one or more immobilized capture ligand such as antibodies, enzymes, nucleic acid aptamers, and the like, for detecting a specific biomarker 190 for which the detection element 188 has a high specificity and affinity. The nano-rods 184 are also coated with crosslinking molecules 186 which immobilize the detection-element molecules 188 onto the nano-rods 184 for capturing and reacting with the corresponding biomarkers 190.

FIG. 3C is a schematic view of the electrochemical-sensor structure 104 showing the substrate 122 and the electrode WE 128′ in some other embodiments. As shown, the electrochemical-sensor structure 104 comprises a nanostructured-sensing surface in the sample-receiving region 134 thereof for amplifying the amount of biomarker binding to the electrochemical-sensor structure 104 in order to achieve improved sensitivity.

In particular, the distal-side electrode WE 128′ comprises a nanostructured-sensing surface 182 having a plurality of capture areas 184′ with ZnO nanomaterials embedded into the capture areas. The capture areas 184′ are coated with a specific type of detection element 188 such as one or more immobilized capture ligand such as antibodies, enzymes, nucleic acid aptamers, and the like, for detecting a specific biomarker 190 for which the detection element 188 has a high specificity and affinity. The capture areas 184′ are also coated with crosslinking molecules 186 which immobilize the detection-element molecules 188 onto the capture areas 184′ for capturing and reacting with the corresponding biomarkers 190.

The electrochemical-sensor structure 104 is engaged with an ex-vivo PoC device 102 which imparts energy to the biological samples and measures the electrochemical properties thereof for generating a reading indicative of the concentration of a specific compound in the biological samples. The volume of the biological samples may be as small as about 10 microliters (µL) to about 20 µL. The imparted energy may be electrical energy and the measured energy property may be the potential difference, current, impedance, and/or the like.

As described above, the analysis circuitry of the portable diagnostic electrochemical-sensing apparatus 102 may be designed corresponding to the circuitry of the electrochemical-sensor structure 104. With the electrochemical-sensor structure 104 shown in FIG. 2E, the analysis circuitry of the portable diagnostic electrochemical-sensing apparatus 102 correspondingly comprises a set of coupling electrodes (e.g., a coupling RE 124, a coupling CE 126, and one or more coupling WEs 128) in the strip-receiving port 112 for electrically engaging the electrodes 124 to 132 of the electrochemical-sensor structure 104.

When biological samples are received into the sample-receiving region 134 of the electrochemical-sensor structure 104, electrochemical interaction (such as oxidization or reduction, depending on the analyte and the detection elements) between the analyte in the biological samples and the detection elements occurs on the WEs 128′ and cause the electrochemical properties to change.

For ease of description, the description of the analysis circuitry below does not differentiate the corresponding REs 124 and 124′, the CEs 126 and 126′, and the WEs 128 and 128′, and may collectively identify the REs, CEs, and WEs using reference numerals 124, 126, and 128, respectively.

The analysis circuitry is configured for measuring one or more impedances, one or more currents, and/or one or more voltages of the first circuitry for analyzing the identified one or more biomarkers in the biological samples. In particular, the analysis circuitry uses RE 124 for measuring and controlling the potentials of the WEs 128, uses CE 126 to provide an excitation signal, and measures the voltage of the response signal at WEs 128. The biological samples on the electrochemical-sensor structure 104 act as electrolyte between WE 128 and CE 126, and sometimes RE 124.

The analysis circuitry comprises one or more potentiostat circuits for electrically coupling to the RE, CE, and WEs 124, 126, and 128 for analyzing one or more biomarkers in the biological samples received in the sample-receiving region 134 of the electrochemical-sensor structure 104. In various embodiments, the one or more potentiostat circuits may comprise a Direct-Current (DC) potentiostat circuit, an Alternate-Current (AC) potentiostat circuit, or a combination thereof.

For example, in some embodiments, the analysis circuitry applies an AC signal at CE 127 and measures the currents WEs 128. After measuring the currents at WEs 128, the analysis circuitry determines the impedance at each WE 128 with respect to the CE 126. The analysis circuitry may vary the frequency of the AC signal within a predefined frequency band (which may be a continuous frequency band or a combination of multiple frequency sub-bands, depending on the implementation) and generates a Nyquist-plot dataset which is then used for determine the patient’s health conditions. Depending on the characteristics of the electrochemical-sensor structure 104 (e.g., the analyte to be detected and the corresponding detection elements), the predefined frequency band may range from sub-hertz (i.e., frequency lower than 1 Hz) to megahertz values.

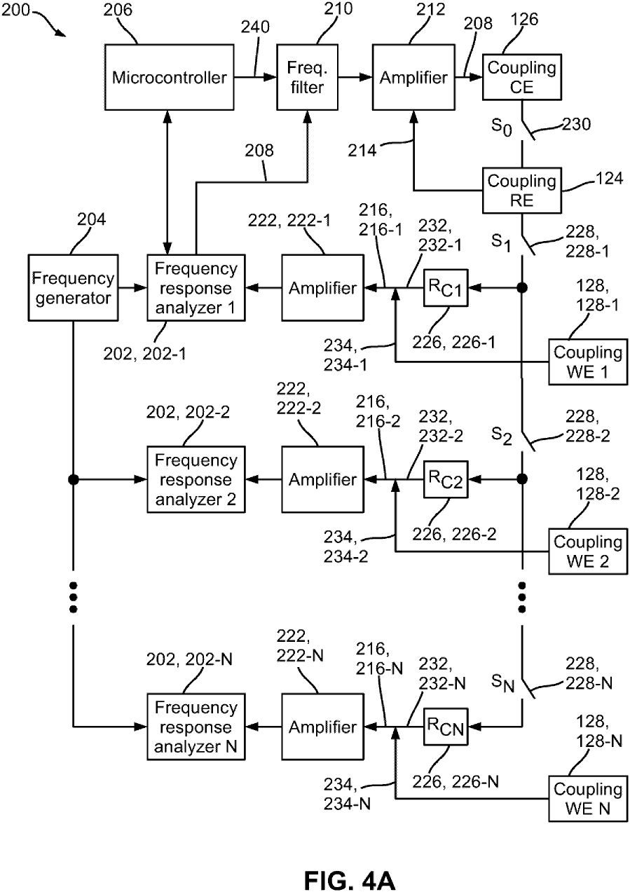

FIG. 4A is a block diagram showing the structure of an analysis circuitry 200 in some embodiments. FIG. 4B shows an example of the analysis circuitry 200.

In these embodiments, the analysis circuitry 200 is in the form of a potentiometer and uses multi-electrode impedance spectroscopy for measuring the electrochemical properties of the biological samples. As shown, the analysis circuitry 200 comprises one or more signal analyzers (FRAs) 202-1, ..., 202-N (where N is the number of coupling WEs 128) such as one or more frequency-response analyzers, in the form of one or more IC chips and receiving a control signal from a frequency generator 204 which generates the control signal of various frequencies within a predefined sweeping frequency-band for “sweeping” the electrochemical-sensor structure 104, that is, applying the generated control signal of various frequencies to the electrochemical-sensor structure 104.

In particular, the frequency generator 204 is connected to the first FRA 202-1 which is in turn connected to a frequency filter 210 and an amplifier 212 for forming an excitation-signal circuit, under the control of a microcontroller 206, to generate an excitation signal 208 and apply the excitation signal 208 to the coupling CE 126. The amplifier 212 also receives a feedback signal 214 from the coupling RE 124.

Herein, each signal analyzer 202 is a component or device for analyzing one or more characteristics of the frequency response of the signal it receives. In some embodiments, the signal analyzer 202 may be a potentiostat for voltammetric, amperometric or potentiometric measurements. For example, in one embodiment, each signal analyzer 202 may be a µStat 400 Bipotentiostat/Galvanostat offered by Metrohm AG of Herisau, Switzerland.

In various embodiments, the frequency filter 210 may be a low-pass filter, a bandpass filter, a notch filter, or a high-pass filter for further shaping the sweeping frequency-band by removing or allowing specific frequency or frequencies (depending on, e.g., the analyte to be detected and the corresponding detection elements).

Each of the FRAs 202-1, ..., 202-N (collectively identified using reference numeral 202) is electrically connected to a respective amplifier 222-1, ..., 222-N.

Each amplifier 222-1, ..., 222-N (collectively identified using reference numeral 222) is electrically connected to a respective coupling WE 128-1, ..., 128-N and a calibration resistor 226-1, ..., 226-N (also denoted R_{C1}, ..., R_{CN}; collectively identified using reference numeral 226) for receiving a signal 216-1, ..., 216-N which may be either a calibration signal 232-1, ..., 232-N from the calibration resistor 226-1, ..., 226-N, or a measurement signal 234-1, ..., 234-N from the coupling WE 128-1, ..., 128-N (described in more detail later). Each calibration resistor 226-1, ..., 226-N is electrically connected to the coupling RE 124 via a respective switch 228-1, ..., 228-N (also denoted S₁, ..., S_{N}; collectively identified using reference numeral 228), which may be, e.g., a gate transistor, a gate semiconductor, or any suitable type. The coupling RE 124 and coupling CE 126 are also electrically connected via a switch 230 (also denoted S₀). The switches 228 and 230 are controlled by the microcontroller 206 to synchronously switch between an OPEN state and a CLOSED state.

Although the switches 228 shown in FIG. 4A are in serial connection to the coupling RE 124, those skilled in the art will appreciate that the switches 228 may alternatively be in parallel connection to the coupling RE 124 or connected to the coupling RE 124 in a mixed connection. As the switches 228 synchronously switchable between the OPEN and CLOSED states, the manner of connection to the coupling RE 124 is not critical.

Although the calibration resistors 226 shown in FIG. 4A are connected to the coupling RE 124 via the switches 228, those skilled in the art will appreciate that the calibration resistors 226 may alternatively connected to the coupling CE 126 via the switches 228.

The analysis circuitry 200 uses the FRAs 202 for determining a Nyquist-plot dataset for calculating the impedances which are then used for determine the patient’s health conditions. In particular, the analysis circuitry 200 employs a two-phase dataset-determination process for determining the Nyquist-plot dataset.

The microcontroller 206 controls both the first FRA 202-1 and the frequency filter 210 for generating the control signal of various frequencies. FIG. 4C shows the detail of the circuit 240 connecting the microcontroller 206 and the frequency filter 210. As shown, the microcontroller 206 comprises a first output pin or terminal 242 connected to a first end of a resistor 244 and a second output pin 248 connected to a first end of a resistor 250. The second ends of the resistors 244 and 250 are connected together and to the first FRA 202-1 and a capacitor 246. In these embodiments, the resistor 250 is similar to the resistor 244. The capacitor 246 is then connected to the frequency filter 210 via a voltage-divider circuit 252 formed by high impedance resistors 254 and 256.

The combination of the high impedance voltage divider 252 and the active component (e.g., the capacitor 246) leads to a longer charge time when the active component 246 is subject to an AC frequency. To enable quick charging of the active component 246, the microcontroller 206 in operation drives the output pins 242 and 248 to a low-impedance high-voltage state (represented in FIG. 4C as “1”) and a low-impedance low-voltage state (represented in FIG. 4C as “0”), respectively, and then drives the output pins 242 and 248 to an INPUT state, thereby allowing quick charging of the active component 246 and subsequently quick activating of the frequency filter 210.

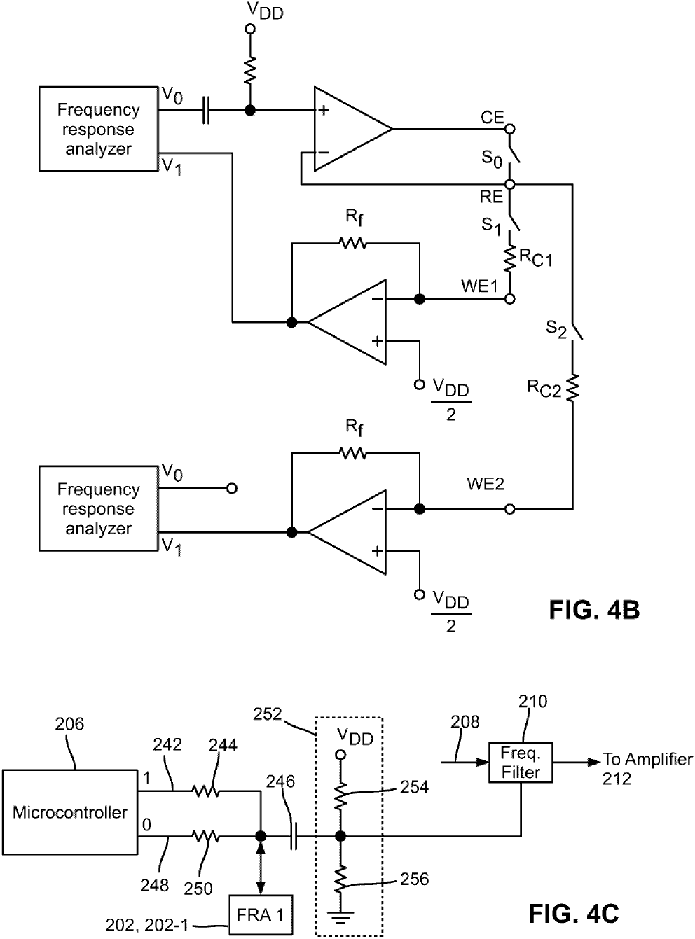

As shown in FIG. 5A, the first phase of the two-phase dataset-determination process is a calibration phase. In this phase, the microcontroller 206 controls the switches 228 and 30 to switch to their CLOSED state thereby short-circuiting or connecting the coupling RE 124 and coupling CE 126, and engaging or connecting the calibration resistors 226 to N calibration circuits for calibrating the parameters of the FRAs 202.

The first calibration circuit involves the first FRA 202-1 applying an excitation signal 208 to the first calibration resistor 226-1 via the frequency filter 210, the amplifier 212, and the short-circuited coupling RE/CE 124/126, and receiving a calibration signal 232-1 from the calibration resistor 226-1 via the amplifier 222-1 (i.e., the signal 216-1 is now the calibration signal 232-1).

Each of the other calibration circuits, e.g., the n-th calibration circuit (n=2, ..., N), involves the first FRA 202-1 applying an excitation signal 208 to the first calibration resistor 226-n via the frequency filter 210, the amplifier 212, and the short-circuited coupling RE/CE 124/126, and the n-th FRA 202-n receiving a calibration signal 232-n from the calibration resistor 226-n via the amplifier 222-n (i.e., the signal 216-n is now the calibration signal 232-n).

As shown in FIG. 5B, the second phase of the two-phase dataset-determination process is a measurement phase. In this phase, the microcontroller 206 controls the switches 228 and 30 to switch to their OPEN state thereby removing the short-circuiting between the coupling RE 124 and coupling CE 126 (i.e., disconnecting the coupling RE 124 and coupling CE 126) and disengaging or disconnecting the calibration resistors 226 therefrom.

An electrochemical-sensor structure 104 with biological samples received in the sample-receiving region 134 thereof is then inserted into the strip-receiving port 112 of the PoC device 102 such that the coupling electrodes 124 to 128 of the PoC device 102 are electrically engaged with the electrode 124 to 128 of the electrochemical-sensor structure 104.

The microcontroller 206 controls the first FRA 202-1 to apply a range of AC frequencies (i.e., varying the frequency of the excitation signal 208; also called sweeping) via the frequency filter 210, amplifier 212, the coupling RE 124 of the PoC device 102, and the RE 124 of the electrochemical-sensor structure 104 to the biological samples.

The FRAs 202-1, ..., 202-N are then measure the voltages of the measurement signals 234-1, ..., 234-N of the respective coupling WEs 128-1, ..., 128-N via the amplifiers 222-1, ..., 222-N for determining the Nyquist-plot datasets for each WE 128-1, ..., 128-N (i.e., the signal 216-n is now the measurement signal 234-n).

In some embodiments, the analysis circuitry 200 does not use the calibration phase (i.e., the first phase). In these embodiments, the process for determining the Nyquist-plot dataset only comprises the measurement phase (i.e., the second phase) and uses pre-measured calibration impedance values for calculating the impedances.

In some embodiments, the calibration resistors 226 may have the same resistance. In some alternative embodiments, the calibration resistors 226 may have different resistances.

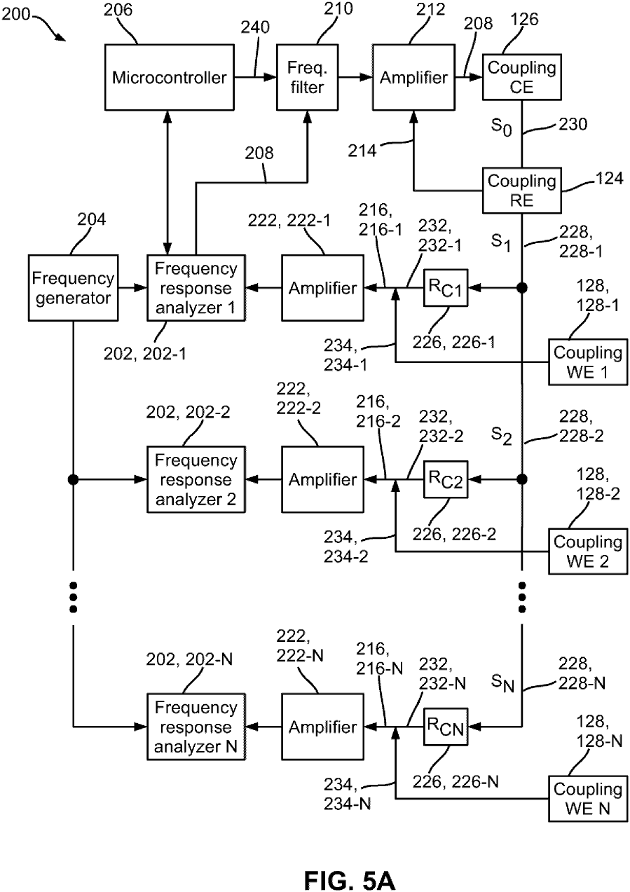

FIG. 6 is a block diagram showing the structure of an analysis circuitry 200 in some alternative embodiments. The analysis circuitry 200 in these embodiments is similar to that shown in FIG. 4A except that in these embodiments, the analysis circuitry 200 only comprises one calibration resistor 226 used by all FRAs 202 during the calibration phase.

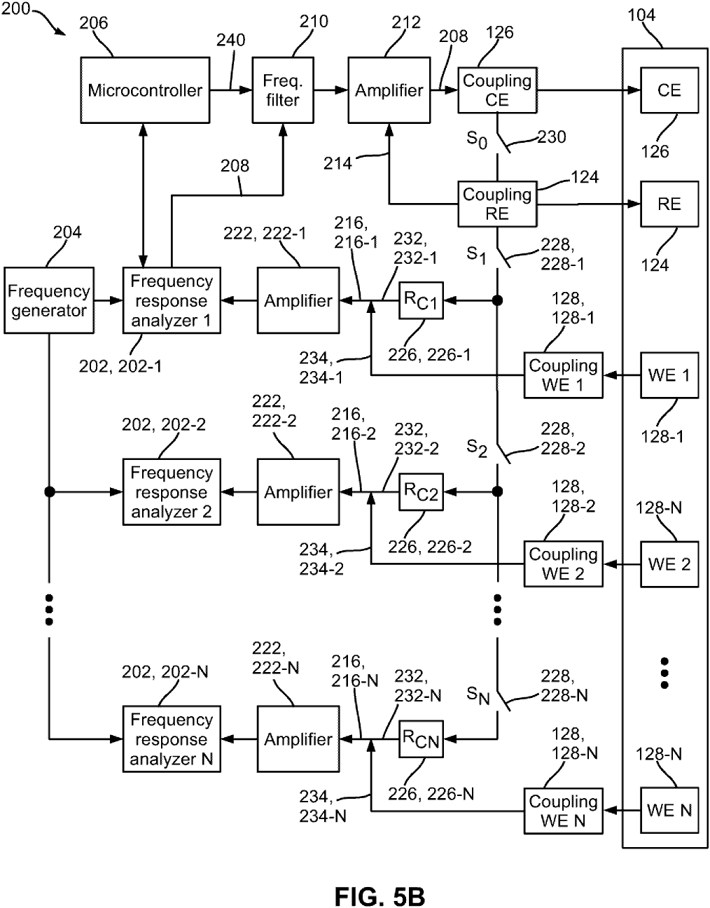

In the embodiments shown in FIGS. 4A and 6, the frequency generator 204 collaborates with the first FRA 202-1 for generating the excitation signal 208. In some alternative embodiments shown in FIG. 7, the frequency generator 204 is connected to the frequency filter 210 for generating the excitation signal 208 without the involvement of the first FRA 202-1. In these embodiments, the function of the first FRA 202-1 is the same as those of other FRAs 202-2, ..., 202N, i.e., determining the Nyquist-plot datasets for each WE 128-1, ..., 128-N.

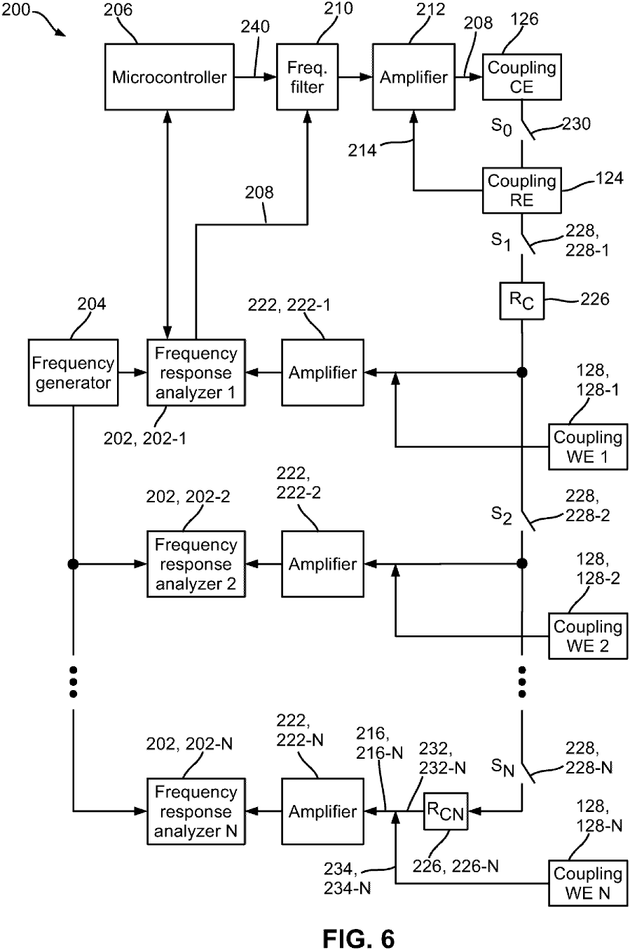

With above-described impedance-determination process, the analysis circuitry 200, which is in the form of a potentiometer, generates a plurality of Nyquist-plot datasets over different complex impedances. As those skilled in the art will appreciate, the impedances are determined under the excitation signal at various frequencies which may cause the electrochemical-sensor structure 104 to respond by changing its impedance thus changing the amount of current flowing through the electrochemical-sensor structure 104, if the electrochemical-sensor structure 104 has undergone any physical changes relating to binding, etching, addition or removal of chemicals, biomolecules or proteins. As shown in FIGS. 8A and 8B, when an excitation signal 208 is applied to the substrate, the response 302 (i.e., the return signal 222 or 232) thereof would generally goes through a transition stage 304 (also denoted a “partial-response stage”), in which the analysis circuitry 200 receives a “partial response” from the electrochemical-sensor structure 104 to its “steady” stage 306 (also denoted a “full-response stage”), in which the analysis circuitry 200 receives a “full response” from the electrochemical-sensor structure 104.

The amount of time taken to scan at a frequency is directly proportional to the inverse of frequency value. For example, to obtain a response from the biological samples, at least one cycle of the excitation AC signal 208 needs to be applied to the sample substrate. The biological samples, on excitation from the single cycle of excitation AC signal 208, result in an emission AC signal 302 (which is a response signal in response to the excitation AC signal 208) whose amplitude would be proportional to the impedance of the substrate. The response signal 302 is then collected into the analysis circuitry 200 for further signal processing.

The time duration to obtain the response signal 302 from the sample substrate depends on the length of each cycle of the excitation AC signal 208. The time duration for each cycle of the excitation AC signal 208 varies inversely to the frequency thereof. For instance, a single cycle of 1000 Hz is 1 ms, a single cycle of 1 Hz is 1 second, a single cycle of 0.1 Hz is 10 seconds, and a single cycle of 0.01 Hz is 100 seconds. Therefore, the minimum time to wait before obtaining the complete response signal 302 from the sample substrate varies directly to the duration of each cycle in the excitation AC signal 208.

Therefore, at lower frequencies, at which the proteins or biomolecules may be the most responsive, the response signal 302 may exhibit a longer duration of the transition stage 304 before reaching the steady stage 306 for proper measurement. As the PoC device 102 may frequency-sweep the electrochemical-sensor structure 104 with low frequencies (e.g., with sub-Hz frequencies in biomolecular detection), the measurement may take a long time thereby preventing quick testing.

In some embodiments, the electrochemical-sensor system 100 uses an artificial intelligence (AI) method such as a machine learning method for predicting the steady-stage measurement data based on a portion of the response signal 302 such as a beginning portion of the response signal 302 before the response signal 302 reaches its steady stage, and uses the predicted data for building the Nyquist-plot dataset. For example, in one embodiment, the electrochemical-sensor system 100 uses a machine learning method for predicting the steady-stage measurement data of lower frequencies (i.e., the steady-stage measurement data in response to an excitation signal 208 of lower frequencies) based on previous impedance measurement of higher frequencies and the known properties of the substrate.

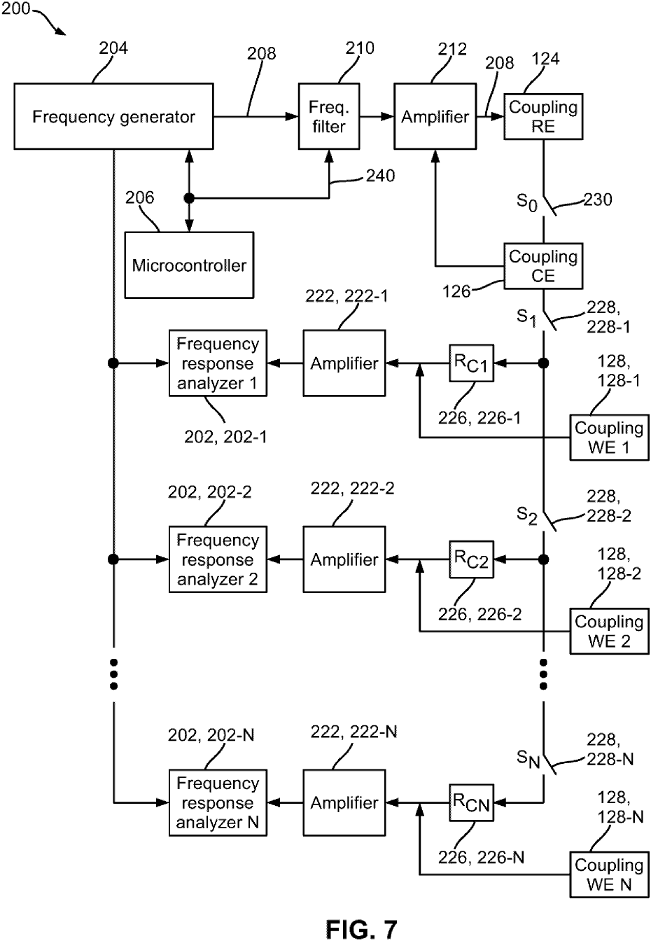

FIG. 9 is a schematic diagram of a process 320 for predicting the steady-stage measurement data using a machine learning method. As shown, a plurality of complete Nyquist-plot datasets 322 are obtained using traditional measurement approach, i.e., by using the analysis circuitry 200 to measure the steady responses of the electrochemical-sensor structure 104 with a frequency-sweeping excitation signal 208. Various complete Nyquist-plot datasets 322 may be obtained for various electrochemical-sensor structures 104.

The obtained complete Nyquist-plot datasets 322 are then fed to a suitable machine-learning module 326 for training an AI prediction engine.

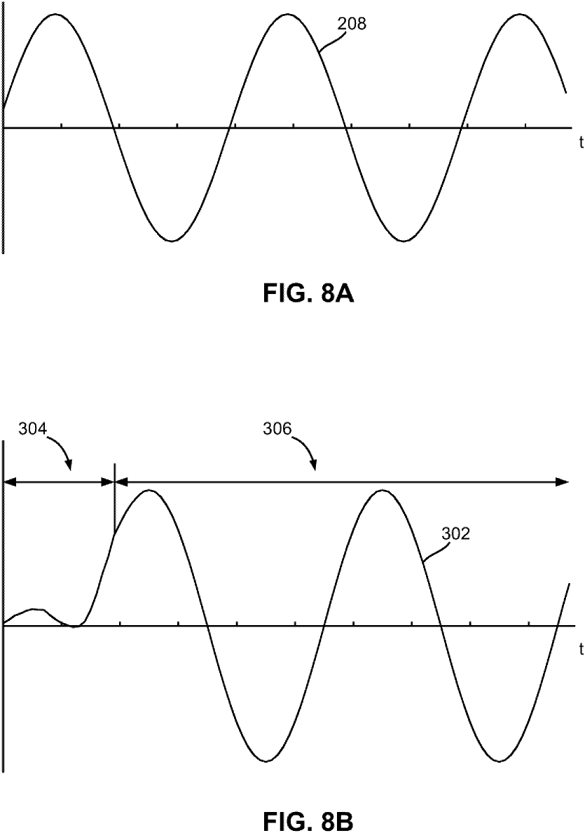

FIG. 10 is a schematic diagram of a deep neural network (DNN) based AI prediction engine for deep learning and for predicting the steady-stage measurement data. As shown, the AI prediction engine comprises a DNN having an input layer 402 with a plurality of input nodes 412, an output layer 406 with a plurality of output nodes 416, and a plurality of cascaded hidden layers 404 intermediate the input and output layers 402 and 406 with each hidden layer 404 having interconnected nodes 414. The hidden layers 404 receive and processes data inputs 412 for generating the outputs 416.

Referring back to FIG. 9, the AI prediction engine is repeated trained using the complete Nyquist-plot datasets 322 and defines impedance values for different frequency ranges as a function of the electrochemical-sensor structures 104, which are used for predicting the steady-stage measurement data.

In use, an input incomplete Nyquist-plot dataset 332 of an electrochemical-sensor structure 104 is obtained. The incomplete Nyquist-plot dataset 332 contain data 334 of a higher-frequency portion (between frequencies f_{A} 336 and f_{B} 338) of the required frequency range. The incomplete Nyquist-plot dataset 332 is then fed to the AI engine for prediction 328 which outputs a complete Nyquist-plot dataset 340 comprising the input incomplete Nyquist-plot dataset 332 of the higher-frequency portion between frequencies f_{A} 336 and f_{B} 338, and the predicted Nyquist-plot dataset 342 of the lower-frequency range between frequencies f_{C} 344 and f_{A} 336.

Those skilled in the art will appreciate that the input incomplete Nyquist-plot dataset 332 of an electrochemical-sensor structure 104 does not have to only comprise data 334 of a higher-frequency portion of the required frequency range. For example, in some embodiments, the input incomplete Nyquist-plot dataset 332 of an electrochemical-sensor structure 104 may comprise impedance data of a lower-frequency portion of the required frequency range and the AI engine may use the input incomplete Nyquist-plot dataset to predict impedance data of lower portion of the required frequency range to output a complete Nyquist-plot dataset comprising impedance data of the entirety of the required frequency range. In some other embodiments, the input incomplete Nyquist-plot dataset 332 of an electrochemical-sensor structure 104 may comprise impedance data of a portion of the required frequency range wherein the portion may be a lower-frequency portion, a mid-frequency portion, a higher-frequency portion of the required frequency range or a mixture thereof. The AI engine may use the input incomplete Nyquist-plot dataset to predict impedance data of other portion of the required frequency range to output a complete Nyquist-plot dataset comprising impedance data of the entirety of the required frequency range.

In above embodiments, the electrochemical-sensing system 100 is a portable system having a portable diagnostic electrochemical-sensing apparatus 102 and a disposable electrochemical-sensor structure 104. In some alternative embodiments, the electrochemical-sensing system 100 may be a desktop or benchtop system having a desktop or benchtop diagnostic electrochemical-sensing apparatus 102 and a disposable electrochemical-sensor structure 104.

In above embodiments, the electrochemical-sensor structure 104 comprises an identification circuitry formed by electrically connected electrodes 130 and 132. In some alternative embodiments, the electrochemical-sensor structure 104 does not comprise any identification circuitry.

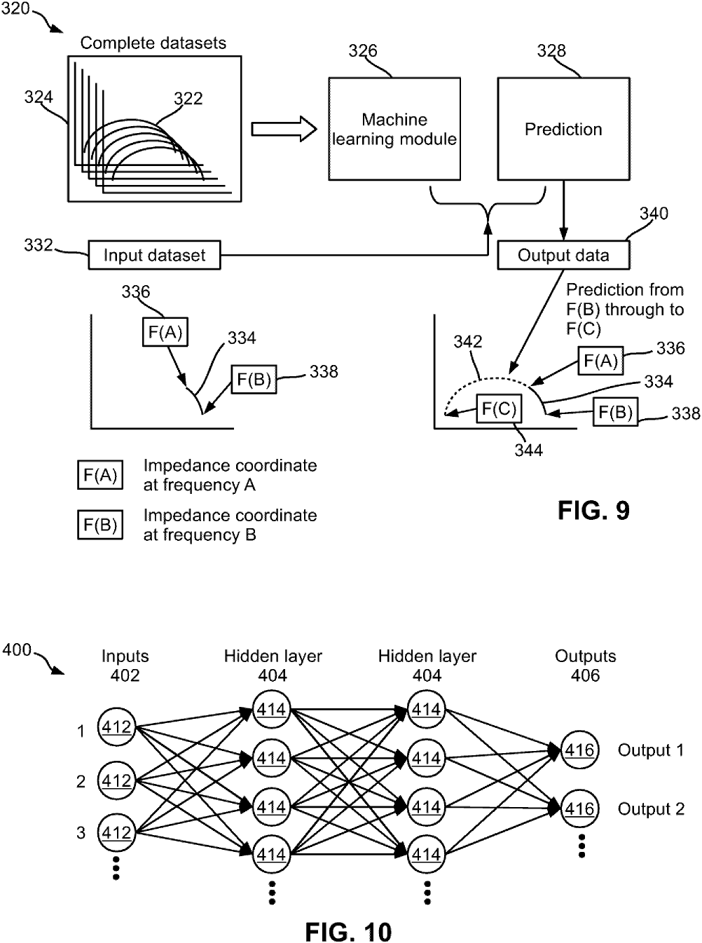

In above embodiments, the prediction of the steady-stage measurement data is performed by the PoC device 102. In some alternative embodiments, the prediction of the steady-stage measurement data may be performed in a separate computing device in communicating with the PoC device 102. FIG. 11 shows the structure of the electrochemical-sensing system 100 in these embodiments.

As shown, the electrochemical-sensing system 500 comprises a server computer 502, a plurality of client-computing devices 504, and one or more health-monitoring data-sources 506 functionally interconnected by a network 508, such as the Internet, a local area network (LAN), a wide area network (WAN), a metropolitan area network (MAN), and/or the like, via suitable wired and wireless networking connections.

Depending on the implementation, the one or more health-monitoring data-sources 506 may comprise one or more personalized health-monitoring or health-data-acquisition devices such as wearable health-monitoring devices 506A (e.g., smartwatches) for collecting patients’ physiological data (such as heart rates, heart rhythms, blood pressures, breathing patterns, blood glucose levels, and/or the like), portable electrochemical-sensing devices 102 described above, portable health-monitoring devices (e.g., portable blood-pressure monitors), and/or similar devices.

The one or more health-monitoring data-sources 506 may alternatively or may also comprise one or more medical records such as medication records 506C collected by patients’ and/or doctors’ computing devices, medical imaging records 506D collected by medical devices of hospitals and/or medical labs, test-result records 506E such as blood test results conducted by hospitals and/or medical labs, and/or the like. Such computing devices and medical devices for obtaining the medical records 506C to 506E may be part of the system 500 in some embodiments. In some other embodiments, the computing devices and medical devices for obtaining the medical records 506C to 506E may not be part of the system 500. Rather, the system 500 provides a data-source interface for interacting with these computing devices and medical devices and receiving medication records 506C to 506E therefrom.

The server computer 502 executes one or more server programs. Depending on implementation, the server computer 502 may be a server-computing device, and/or a general-purpose computing device acting as a server computer while also being used by a user.

The client-computing devices 504 include one or more client-computing devices 504A used by one or more patients and one or more client-computing devices 504B used by doctors. Each client-computing device 504 executes one or more client application programs (or so-called “Apps”) and for users to use. The client-computing devices 504 may be portable computing devices such as laptop computers, tablets, smartphones, Personal Digital Assistants (PDAs) and the like. However, those skilled in the art will appreciate that one or more client-computing devices 504 may be non-portable computing devices such as desktop computers in some alternative embodiments.

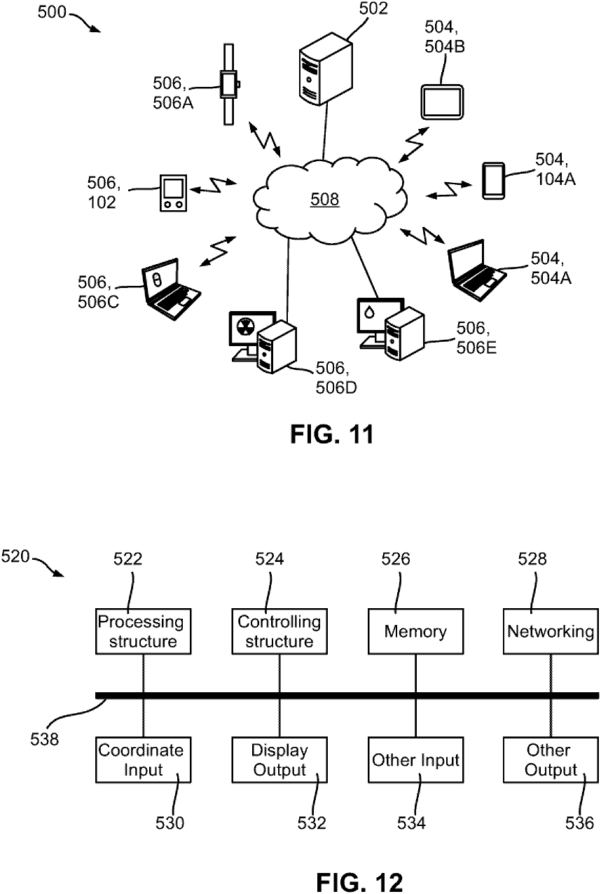

Generally, the computing devices 502 and 504 have a similar hardware structure such as a hardware structure 520 shown in FIG. 12. As shown, the computing device 502/504 comprises a processing structure 522, a controlling structure 524, memory or storage 526, a networking interface 528, coordinate input 530, display output 532, and other input and output modules 534 and 536, all functionally interconnected by a system bus 538.

The processing structure 522 may be one or more single-core or multiple-core computing processors such as INTEL® microprocessors (INTEL is a registered trademark of Intel Corp., Santa Clara, CA, USA), AMD® microprocessors (AMD is a registered trademark of Advanced Micro Devices Inc., Sunnyvale, CA, USA), ARM® microprocessors (ARM is a registered trademark of Arm Ltd., Cambridge, UK) manufactured by a variety of manufactures such as Qualcomm of San Diego, California, USA, under the ARM® architecture, or the like.

The controlling structure 524 comprises one or more controlling circuits, such as graphic controllers, input/output chipsets and the like, for coordinating operations of various hardware components and modules of the computing device 502/504.

The memory 526 comprises a plurality of memory units accessible by the processing structure 522 and the controlling structure 524 for reading and/or storing data, including input data and data generated by the processing structure 522 and the controlling structure 524. The memory 526 may be volatile and/or non-volatile, non-removable or removable memory such as RAM, ROM, EEPROM, solid-state memory, hard disks, CD, DVD, flash memory, or the like. In use, the memory 526 is generally divided to a plurality of portions for different use purposes. For example, a portion of the memory 526 (denoted as storage memory herein) may be used for long-term data storing, for example, storing files or databases. Another portion of the memory 526 may be used as the system memory for storing data during processing (denoted as working memory herein).

The networking interface 528 comprises one or more networking modules for connecting to other computing devices or networks through the network 508 by using suitable wired or wireless communication technologies.

The display output 532 comprises one or more display modules for displaying images, such as monitors, LCD displays, LED displays, projectors, and the like. The display output 532 may be a physically integrated part of the computing device 502/504 (for example, the display of a laptop computer or tablet), or may be a display device physically separate from, but functionally coupled to, other components of the computing device 502/504 (for example, the monitor of a desktop computer).

The coordinate input 530 comprises one or more input modules for one or more users to input coordinate data, such as touch-sensitive screen, touch-sensitive whiteboard, trackball, computer mouse, touch-pad, or other human interface devices (HID) and the like. The coordinate input 530 may be a physically integrated part of the computing device 502/504 (for example, the touch-pad of a laptop computer or the touch-sensitive screen of a tablet), or may be a display device physically separate from, but functionally coupled to, other components of the computing device 502/504 (for example, a computer mouse). The coordinate input 530, in some implementation, may be integrated with the display output 532 to form a touch-sensitive screen or touch-sensitive whiteboard.

The computing device 502/504 may also comprise other input 534 such as keyboards, microphones, scanners, cameras, Global Positioning System (GPS) component, and/or the like. The computing device 502/504 may further comprise other output 536 such as speakers, printers and/or the like.

The system bus 538 interconnects various components 522 to 536 enabling them to transmit and receive data and control signals to/from each other.

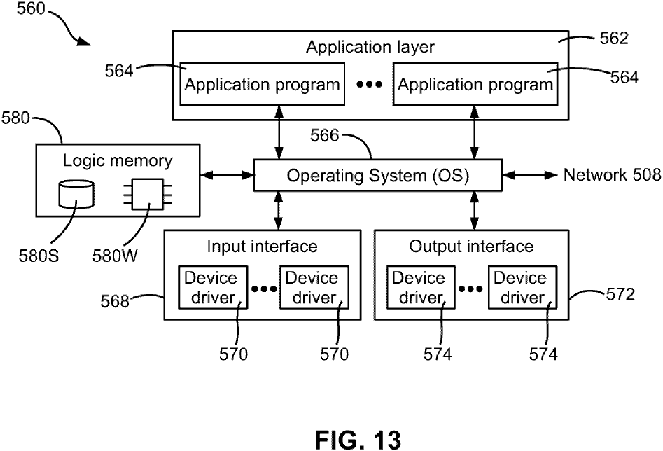

FIG. 13 shows a simplified software architecture 560 of the computing device 502 or 504. The software architecture 560 comprises an application layer 562, an operating system 566, an input interface 568, an output interface 572, and logic memory 580. The application layer 562 comprises one or more application programs 564 executed by or run by the processing structure 522 for performing various tasks. The operating system 566 manages various hardware components of the computing device 502 or 504 via the input interface 568 and the output interface 572, manages logic memory 580, and manages and supports the application programs 564. The operating system 566 is also in communication with other computing devices (not shown) via the network 508 to allow application programs 564 to communicate with those running on other computing devices. As those skilled in the art will appreciate, the operating system 566 may be any suitable operating system such as MICROSOFT® WINDOWS® (MICROSOFT and WINDOWS are registered trademarks of the Microsoft Corp., Redmond, WA, USA), APPLE® OS X, APPLE® iOS (APPLE is a registered trademark of Apple Inc., Cupertino, CA, USA), Linux, ANDROID® (ANDROID is a registered trademark of Google Inc., Mountain View, CA, USA), or the like. The computing devices 502 and 504 of the personalized health-monitoring system 500 may all have the same operating system, or may have different operating systems.

The input interface 568 comprises one or more input device drivers 570 for communicating with respective input devices including the coordinate input 530. The output interface 572 comprises one or more output device drivers 574 managed by the operating system 566 for communicating with respective output devices including the display output 532. Input data received from the input devices via the input interface 568 is sent to the application layer 562, and is processed by one or more application programs 564. The output generated by the application programs 564 is sent to respective output devices via the output interface 572.

The logical memory 580 is a logical mapping of the physical memory 526 for facilitating the application programs 564 to access. In this embodiment, the logical memory 580 comprises a storage memory area (580S) that is usually mapped to non-volatile physical memory such as hard disks, solid-state disks, flash drives, and the like, generally for long-term data storage therein. The logical memory 580 also comprises a working memory area (580W) that is generally mapped to high-speed, and in some implementations volatile, physical memory such as RAM, generally for application programs 564 to temporarily store data during program execution. For example, an application program 564 may load data from the storage memory area 580S into the working memory area 580W, and may store data generated during its execution into the working memory area 580W. The application program 564 may also store some data into the storage memory area 580S as required or in response to a user’s command.

In a server computer 502, the application layer 562 generally comprises one or more server-side application programs 564 which provide server functions for managing network communication with client-computing devices 504 and facilitating collaboration between the server computer 502 and the client-computing devices 504. Herein, the term “server” may refer to a server computer 502 from a hardware point of view or a logical server from a software point of view, depending on the context.

In these embodiments, the server-side application programs 564 comprises an analysis program or program module (also identified using reference numeral 564, which may be considered as a part of the analysis circuitry) for executing the process 320 for predicting the steady-stage measurement data. The analysis program 564 executes the process 320 to collect a plurality of complete Nyquist-plot datasets 322 from one or more PoC devices 102 for training the AI prediction engine. When a PoC device 102 obtains an input incomplete Nyquist-plot dataset 332 of an electrochemical-sensor structure 104, the PoC device 102 sends the incomplete Nyquist-plot dataset 332 to the analysis program 564 for predicting the steady-stage measurement data of low frequencies. After prediction, the analysis program 564 sends the complete Nyquist-plot dataset 340 back to the PoC device 102.

In above embodiments, the switches 228 are synchronously switchable between the OPEN and CLOSED states. In some embodiments, the switches 228 may not all be synchronously switchable (e.g., some of the switches 228 may be asynchronously switchable with respect to others of the switches 228). In these embodiments, the above-described calibration and measurement operations may be conducted after all switches 228 are switched to the CLOSED state or the OPEN state.

In above embodiments, the calibration resistors 226 are connected to the FRAs 202, and the switches 228 are switchable between the OPEN and CLOSED states to disconnect or connect, respectively, the calibration resistors 226 to the coupling CE 126 and coupling RE 124. In some embodiments, the calibration resistors 226 are connected to the coupling CE 126 or the coupling RE 124, and the switches 228 may be switchable between the OPEN and CLOSED states to disconnect or connect, respectively, the calibration resistors 226 to the FRAs 202.

In some embodiments, the analysis circuitry 200 may not comprise a plurality of frequency generators 204 for providing control signals to the FRAs 202.

In some embodiments, each FRA 202 may comprise its own frequency generator 204.

In some embodiments, the analysis circuitry 200 may comprise a frequency generator 204 connecting to some of the FRAs 202, and other FRAs 202 may each comprise or otherwise integrate in its own frequency generator.

Although embodiments have been described above with reference to the accompanying drawings, those of skill in the art will appreciate that variations and modifications may be made without departing from the scope thereof as defined by the appended claims.

1. A circuitry for analyzing one or more biomarkers in a sample of a user, the circuitry comprising:
a coupling counter electrode (CE), a coupling reference electrode (RE), and one or more coupling working electrodes (WEs);
an excitation-signal circuit for generating an excitation signal and applying the excitation signal to the coupling CE and the coupling RE;
one or more signal analyzers each electrically connected to a respective one of the one or more coupling WEs for receiving a return signal from the respective coupling WE in response to the excitation signal for analyzing the one or more biomarkers;
at least one calibration resistor; and
a set of switches each switchable between an OPEN state and a CLOSED state;
wherein the set of switches are configured for, when in the CLOSED states, electrically connecting the coupling CE and the coupling RE and electrically connecting the one or more signal analyzers to the connected coupling CE and coupling RE via the at least one calibration resistor for directing a calibration signal to the at least one frequency analyzer component for calibration; and
wherein the set of switches are configured for, when in the OPEN states, electrically disconnecting the coupling CE from the coupling RE and electrically disconnecting the one or more signal analyzers from the coupling CE and the coupling RE for analyzing the one or more biomarkers.

2. The circuitry of claim 1, wherein the set of switches are synchronously switchable between the OPEN state and the CLOSED state.

3. The circuitry of claim 1, wherein the at least one calibration resistor is a single calibration resistor.

4. The circuitry of claim 1, wherein the at least one calibration resistor comprise a plurality of calibration resistors.

5. The circuitry of claim 4, wherein the plurality of calibration resistors have a same resistance, or at least a first subset and a second subset of the plurality of calibration resistors have different resistances.

6. (canceled)

7. The circuitry of claim 1 , wherein the one or more signal analyzers are electrically connected to the one or more coupling WEs via one or more first amplifiers with each signal analyzer electrically connected to the respective WE via a respective one of the one or more first amplifiers.

8. The circuitry of claim 7, wherein the set of switches are configured for, when in the CLOSED state, electrically connecting the coupling CE and the coupling RE and electrically connecting the one or more first amplifiers to the connected coupling CE and coupling RE via the at least one calibration resistor.

9. The circuitry of claim 1 further comprising:
at least one first frequency generator for providing one or more control signals to the one or more signal analyzers.

10. The circuitry of claim 9, wherein the at least one first frequency generator is configured for generating the one or more control signals of various frequencies within a predefined sweeping frequency-band.

11. The circuitry of claim 1 , wherein the excitation-signal circuit comprises a second frequency generator for generating the excitation signal.

12. The circuitry of claim 11, wherein the second frequency generator comprises a first one of the one or more frequency-response analyzers.

13. The circuitry of claim 11 , wherein the excitation-signal circuit further comprises a frequency filter for filtering an output of the second frequency generator for generating the excitation signal.

14. The circuitry of claim 13, wherein the excitation-signal circuit further comprises a microcontroller for controlling the frequency filter and the second frequency generator.

15. The circuitry of claim 13 , wherein the excitation-signal circuit further comprises a second amplifier for amplifying an output of the frequency filter for generating the excitation signal.

16. An electrochemical-sensing apparatus for analyzing a sample of a user, the apparatus comprising:
an analysis circuitry of claim 1 ; and
an output for outputting an analytical result of said analysis of the one or more biomarkers in the sample.

17. The electrochemical-sensing apparatus of claim 16 further comprising:
a housing comprising at least one first port for receiving an electrochemical-sensor structure, the electrochemical-sensor structure comprising a first set of electrodes for contacting the sample, the first set of electrodes comprising a CE for coupling with the coupling CE, a RE for coupling with the coupling RE, and one or more WEs for coupling with the one or more coupling WEs.

18. An electrochemical-sensing system for analyzing a sample of a user, the system comprising:
an analysis circuitry for applying to the sample an excitation signal sweeping a predefined first frequency range and receiving a response signal for analyzing one or more biomarkers in the sample;
an output for outputting an analytical result of said analysis of the one or more biomarkers in the sample; and
a prediction module for using an artificial-intelligence (AI) method for predicting a response signal in response of the excitation signal sweeping a predefined second frequency range.

19. The electrochemical-sensing system of claim 18, wherein the AI method comprises a deep neural network (DNN).

20. The electrochemical-sensing system of claim 18 , wherein the second frequency range is lower than the first frequency range.

21. The electrochemical-sensing system of claim 18 further comprising:
an electrochemical-sensing apparatus comprising a housing receiving therein the analysis circuitry, the housing comprising a display for displaying the output and at least one first port for receiving an electrochemical-sensor structure, the electrochemical-sensor structure comprising a first set of electrodes for contacting the sample, the first set of electrodes comprising a CE for coupling with the coupling CE, a RE for coupling with the coupling RE, and one or more WEs for coupling with the one or more coupling WEs.

22. (canceled)

23. The electrochemical-sensing system of claim 21, wherein the the prediction module is received in the housing of the electrochemical-sensing apparatus, or is in a computing device configured for communicating with the electrochemical-sensing apparatus.

24. An electrochemical-sensing system for analyzing a sample of a user, the system comprising:
an analysis circuitry for applying to the sample an excitation signal sweeping a predefined first frequency range and receiving a response signal for analyzing one or more biomarkers in the sample;
an output for outputting an analytical result of said analysis of the one or more biomarkers in the sample; and
a prediction module for using an AI method for predicting steady-stage measurement data based on a portion of the response signal.

25. The electrochemical-sensing system of claim 24, wherein the portion of the response signal is a beginning portion of the response signal before the response signal reaches a steady stage.

26. (canceled)
